(19) United States
(12) Patent Application Publication
(10) Pub. No.: US 2004/0241129 A1
(43) Pub. Date: Dec. 2, 2004

(54) Amphiphilic complexes, method for their preparation and compositions containing them

(75) Inventors: Eric Perrier, Les Cotes d'Arey (FR); Alain Huc, Ste. Foy les Lyon (FR); Danielle Antoni, Vernaison (FR); Coralie Roussel, Corconne (FR); Michel Pina, Montpellier (FR); Jean Graille, Montpellier (FR)

Correspondence Address: MERCHANT & GOULD PC, P.O. BOX 2903, MINNEAPOLIS, MN, 55402-0903, US

(73) Assignee: COLETICA, Lyon (FR)

(21) Appl. No.: 10/795,602

(22) Filed: Mar. 8, 2004

Related U.S. Application Data
(63) Continuation-in-part of application No. 08/877,124, filed on Jun. 17, 1997, now Pat. No. 6,703,490.
Application No. 08/877,124 is a continuation of application No. PCT/FR96/01620, filed on Oct. 17, 1996.

(30) Foreign Application Priority Data
Oct. 17, 1995 (FR) 95 12137

(51) Int. Cl. A61K 7/06; C07K 14/00

(52) U.S. Cl. 424/70.14; 530/410

(57) ABSTRACT

The invention relates to novel amphiphilic complexes, a method for their preparation, and the compositions containing them. Said amphiphilic complexes result from the reaction, at a temperature between ambient temperature and 80° C., between a protein or polypeptide whose average molecular mass is greater than or equal to 5,000 Daltons and a fatty chain whose carbon atom number is between 4 and 30 selected from fatty acid, fatty alcohol, fatty amine, with the exclusion of undecylenic acid ; the protein or polypeptide/fatty chain weight ratio ranging from 1000/1 to 1/10.

This application is a Continuation-in-Part of U.S. Ser. No. 08/877,124, under allowance to be issued on March 9, 2004 as U.S. Pat. No. 6,703,490, which is itself a US national phase of PCT/FR96/01620 having an International Filing Date of Oct. 16, 1996.

The first object of the present invention is amphiphilic (hydrolipidic) complexes and more specifically proteins (and polypeptides) onto which fatty chains have been grafted. Said complexes may be qualified lipophilised proteins (and polypeptides). Other objects of the present invention are compositions, notably cosmetic, pharmaceuticals or food compositions which contain such complexes and methods for the preparation of said complexes.

The skin may be considered as an organ which separates and protects the human body from its environment. This effect of a barrier against external damaging effects is capital in order that the internal tissues suitably exert their function. External damaging effects are in fact many: luminous damaging effects (UVA, UVB, infra-red) which cause free radicals and fragmentation of the constituents of the skin, physical or mechanical damaging effects (abrasions, variations in temperature and hygrometry . . . ) which cause inflammations, chemical damaging effects (air and water pollution, contact with irritant or immunogenic elements), microbiological damaging effects (bacteria, viruses, fungi . . . ). In order to react to these various damaging effects, the skin possesses a certain number of specialised cells which sometimes form extremely well 20 characterised structures. This is the case of the corneocytes which, being different from keratinocytes, form a structure called the Stratum corneum which is specialised in the protection of the most internal areas of the skin. This superficial horny structure is the first protection against external damaging effects.

The use of cosmetic products and notably of hydration products also comes up against this natural barrier:

due to their small size, hydrophilic molecules of low molecular mass such as urea, lactic acid, amino acids, can penetrate via the Stratum corneum as far as in the deepest layers of the cutaneous tissues. The cosmetic effect obtained is a hydrating effect upon the deep layers of the epidermis and the dermis, an effect which is relatively short lived;

on the contrary, molecules of higher molecular mass such as proteins for example cannot cross this barrier. The Stratum corneum is in fact principally constituted of lipids (its lipid content neighbours 80% by weight), giving it a particularly hydrophobic character, totally incompatible with the hydrophilic character of most proteins used in the cosmetology field. In this case, the cosmetic effect obtained is a filmogenic effect, at times interesting for obtaining particular textures or “cosmetic feels” but which remains totally and exclusively superficial.

Thus, and consequently, the hydrophilic molecules used to this day in cosmetics are cast aside by this hydrophobic structure and either stay on the surface or penetrate the dermis very deeply. From this, the horny layer and the upper layers of the epidermis are under practically no influence of the active and notably hydrating substances used up to this day in cosmetics. Now, the feeling of dryness of the skin comes from the Stratum corneum and upper layers of the epidermis. It is therefore of utmost importance to manage efficiently hydrating this structure and more generally to render said structure accessible to various hydrophilic entities.

The Applicant, within the context of the present invention, has taken on this technical problem of hydration of the skin and more generally that of the optimisation of the expression of the activity of molecules of the protein or polypeptide type upon the Stratum corneum. In order to solve said technical problem, the Applicant proposes modifying the physico-chemical character of said molecules and to thus modify the behaviour of it. The Applicant proposes in fact to generate amphiphilic complexes by grafting fatty chains onto said molecules. The trans-epidermal penetrations of such complexes are different from those of the non-complexed molecules. Their stabilisation in the upper layers of the epidermis as well as on the capillary fibre (hair) has been demonstrated. Furthermore, very interesting and unexpected cosmetic even therapeutic effects of said complexes have been observed.

It has been described in the patent application FR-A-2 671 725 about polyose-fatty acid complexes which have hydrating and emulsifying properties. These complexes are obtained by reacting, in aqueous medium, at ambient temperature, fatty acids in a reactive form with polyoses. Said polyoses can intervene in an impure form and notably in a mixture with proteins. However, in this document, no mention is made of a “binary” protein-fatty acid complex and of the interesting properties it could have . . . In any case, the polysaccharides (polyoses) having a gellifying power much grater than that of proteins, obtaining hydrating and emulsifying complexes by lipophilising such proteins could not be expected. Such is ail the same one of the results obtained within the context of the present invention . . .

It has also been described:

in the patent US-A-4,234,475 a method of preparing emulsifying agents which consists in reacting, at temperatures above 200° C., a protein and an acid, notably a fatty acid. At such temperatures, the degradation of each one of the reagents cannot be prevented, and notably the degradation of the protein (denatured and/or hydrolysed into peptides), whose properties are consequently inescapably altered;

in the application WO-A-93 22370 undecylenic acid derivatives obtained by reacting said acid in a reactive form, in an aqueous medium, at ambient temperature with a hydrophilic organic macromolecule having primary alcohol groups and/or primary amine groups, and notably with a protein. Said derivatives, very slightly fragrant, have conserved anti-fungal and anti-bacterial properties. By their approach, the expression of the activities of undecylenic acid have been above all sought-after.

Furthermore:

the application DE-A-34 22 496 describes an alcoholic disinfectant composition for the skin. Said composition contains a protein hydrolysate, a mixture of amino acids in fact,

the application EP-A-0 417 619 proposes, as a detergent showing a lesser agressivity towards the skin and the mucous membranes, the condensation products resulting from the chemical reaction between:

a hydrolysate of proteins whose average molecular mass is between 3,000 and 7,000; and

a C₁₂-C₁₈ fatty acid; said chemical reaction being carried out at a pH between 7 and 12 and the protein(s)/fatty acid(s) molar ratio ranging from 1/0.5 to 1/3;

the application EP-A-0 283 601 describes elastin derivatives prepared from hydrolysed elastin. Said derivatives result from a chemical coupling between said hydrolysed elastin (non-native) and a fatty acid anhydride; said fatty acid intervening, with respect to the protein (hydrolysed elastin) in a weight ratio very much lower than 1.

Said condensation products according to EP-A-0 417 619 and elastin derivatives according to EP-A-0 283 601 are excluded and are not complexes within the sense of the invention. Said complexes of the invention can be elaborated with native proteins, in particular plant proteins. This is explained below.

The Applicant in fact proposes novel amphiphilic or hydrolipidic complexes—protein(s)/fatty chain(s) complexes—which, as indicated above, have very interesting and relatively unexpected cosmetic even therapeutic properties.

It is herein specified that, in the present text—within the context of the present invention—the term protein is used to designate a “real” protein as well as a polypeptide (obtained eventually by synthesis or as well known by hydrolysis of proteins).

Said complexes of the invention are, in a characteristic way, obtained from the reaction carried out at a temperature between ambient temperature and 80° C. between:

on the one hand, one (or more) protein(s) or polypeptide(s) whose average molecular mass is greater than or equal to 5,000 Daltons; and

on the other hand, one (or more) fatty chain(s), whose carbon atom number is between 4 and 30, selected from fatty acids, fatty alcohols, fatty amines and derivatives thereof, with the exclusion of undecylenic acid when the fatty acid is in a weight ratio in excess to the protein, the [protein or polypeptide/fatty chain] weight ratio may vary between very wide limits ranging from 1000/1 to 1/10, namely from a very large excess in protein to an excess in fatty chain.

In an advantageous embodiment, notably of interest when the fatty chain is grafted with a monofunctional grafting agent which may be the fatty containing component (fatty monoacid, monoalcohol or monoamine) under an appropriate form or another monofunctional grafting agent, the [protein(s)/fatty chain(s)] weight ratio is ranging from 1/1 to 1/10 and advantageously from 1/3 to 1/5.

According to another advantageous embodiment, notably of interest when the fatty chain is grafted with a multifunctional grafting agent which may be the fatty containing component (fatty polyacid, polyalcohol or poly amine) under an appropriate form or another polyfunctional grafting agent, the [protein(s)/fatty chain(s)] weight ratio is ranging from 1000/1 to 1/3 and advantageously from 1000/1 to 10/1.

The reaction carried out for the coupling and/or grafting of the reagents may be chemical or enzymatic. This shall be specified further on in the present text. In any case, the reaction is carried out at a temperature much lower than 200° C., lower than 100° C. The minimisation even prevention of any degradation of the reagents and notably the intervening proteins is desired.

Said reaction is carried out with two types of reagent: on the one hand, at least one protein, on the other hand at least one fatty chain. Said fatty chains consist of fatty acids as well as fatty alcohols or fatty amines (or the derivatives of said acids, alcohols and amines).

The multiplicity and the variety of the complexes of the invention may already be insisted upon and therefore the properties that they may have; the latter depending upon the nature of the reagents (intervening protein(s) and fatty chain(s) and of their intrinsic characteristics (for example, the nature of the intervening protein, the purity of it, the molecular weight of it).

Each one of both types of reagent is specified below.

The complexes of the invention are complexes comprising the reaction product of proteins and fatty chains. The complexes obtained from amino acids are excluded from the context of said invention, whether they be purified or obtained in a mixture during the hydrolysis of a protein, as well as the complexes obtained from peptides having only 2 to 5 amino acids in their structure. The proteins or polypeptides which can intervene in the structure of the complexes of the invention have an average molecular mass equal to or greater than 5,000 Daltons. They consist of a chain of amino acids linked to each other by amide bonds, which has pendant amine and/or acid and/or alcohol functions.

Generally, their average molecular mass is lower than 1,000,000 Daltons. It is however in no way excluded to prepare complexes of the invention with proteins of a greater average molecular mass. Advantageously, the complexes of the invention are prepared from proteins whose average molecular mass is between 10,000, preferably between 20,000, and 1,000,000 Daltons. Even more advantageously, proteins whose average molecular mass is between 20,000 and 300,000 Daltons are brought in.

In any case, the intervening proteins can be obtained by an extraction which does not destroy their structure and/or does not lower their molecular mass, or by moderate physical, chemical or enzymatic hydrolysis (said hydrolysis generating proteins whose average molecular mass is at least equal to 5,000 Daltons).

Said intervening proteins may be of animal origin (bovine, ovine, fish, shark, crustaceans, . . . ) and in this case, they may be extracted from various tissues; collagen, gelatine, albumin, ovalbumin, elastin, reticulin, fibronectin, keratin, silk, laminin, desmosin and isodesmosin, extracellular matrix proteoglycans, caseins, lactalbumin, lactoglobulins, enzymes extracted from animal tissues, etc . . . may be cited by as examples.

The proteins may be of plant origin such as marine plants unicellular or multicellular algae ; ground plants comprising the leguminous plant, cereals like wheat, moderated wheat, maize, barley, malt, oat, cotton, lupin (genus Lupinus), soya (genus Glycine), pea (genus Pisum), chick pea (Cicer), lucerne (Medicago), horse bean (Vicia), lentil (Lens), bean (Phaseolus), colza (Brassica), sunflower (Helianthus), broad bean, almond, bean.

The proteins and hydrolysates or polypeptides may be extracted from seeds, flowers, fruits, barks, gums, etc . . . ;

Advantageously the plant proteins are extracted from cereals. Preferably the cereal is selected from the group consisting of wheat, maize, barley, malt, soya, oat, Lucerne, lupin, lentil, colza.

The plant proteins may be industrially used in the form of pulverulent preparations selected from the group consisting of flours, concentrates and isolates, and liquid preparations. Industrial well known liquid preparations are soya milks.

The plant proteins are advantageously dissolved or dispersed at a concentration of between 0.5 and 5% by weight in an aqueous solution with a pH of between about 4.5 and about 8.

As regards the second type of reagent, these are as already specified, fatty chains having from 4 to 30 carbon atoms. Advantageously, the intervening fatty chains have from 6 to 20 carbon atoms. The chains may be saturated or unsaturated, linear, branched or cyclic. They obviously have acid and/or alcohol and/or amine functions but it is in no way excluded that they have other chemical functions in their structure which intervene or do not intervene in the preparation of the complexes of the invention.

It has been seen that said fatty chains consist of fatty acids, fatty alcohols or fatty amines (or their derivatives) which may be monofunctional or polyfunctional. For the preparation of the complexes of the invention chemically, notably said fatty acids optionally intervene in reactive forms (more reactive), and notably as halides (chlorides, bromides, iodides, . . . ), anhydrides or derivatives of anhydrides.

According to a first embodiment, said fatty chains may notably be selected from the heptanoic, octanoic, decanoic, lauric, rnyristic, palmitic, stearic, ricinoleic, oleic, linoleic, linolenic fatty acids; the corresponding fatty alcohols and fatty amines; the derivatives of said fatty acids, fatty alcohols and fatty amines; and mixtures thereof.

Advantageously, complexes of the invention are prepared:

with lauric, stearic or palmitic acids and notably a mixture of stearic and palmitic acids;

with laurylamine or hexadecylamine;

with decylalcohol.

It may be observed from this first embodiment that this first list of fatty containing components comprises mono reactive functions: monoacid, monoalcohol, monoamine and mixtures thereof.

Within the structure of the complexes of the invention under this first embodiment, the [protein(s)]/[fatty chain(s)] weight ratio ranges from 1/1 to 1/10 and preferentially from 1/3 to 1/5, thereby providing an excess of fatty chains.

The protein(s) are in fact in this first embodiment allowed to react with a more or less large excess of fatty chain(s) with the aim of creating covalent bonds but also of the ionic, hydrogen and Van der Waals type.

According to a second embodiment, the fatty chains are grafted with a multifunctional grafting agent which may be the fatty containing component under an appropriate form (fatty polyacid, polyalcohol or polyamine) or another polyfunctional grafting agent. In such a case it is preferred that the [protein(s)/fatty chain(s)] weight ratio is ranging from 1000/1 to 1/3 and advantageously from 1000/1 to 10/1, namely usually a large molar excess of protein .

The polyfunctional grafting agent is preferably provided by the fatty chains which bear at least two functions, i.e. functional groups, selected from acid or anhydrid, alcohol or amine, and any mixture thereof. Examples of said polyfunctional agents are: phtaloyl, terephtaloyl, sebacoyl, glutaryl, adipoyl or succinyl acids, preferably under reactive form as dihalides or anhydrides. It is further preferable to use the dichloride of these acids. Of course tri- and further polyfunctional grafting agents (for instance citric acid) can be used and one skill in the art will know to set the relative proportion between protein and the polyfunctional grafting agent in view of the present disclosure taken as a whole with the enabling examples.

Generally and furthermore, at the end of the reaction, the unreacted fatty chains which are not bound to the protein are not recovered, the isolation of “binary” complexes of the pure protein(s)-fatty chains type is not attempted. Thus, the complexes of the invention generally consist of “binary” complexes of the type indicated above in a mixture with non-bound fatty chains; in other words, they consist of the product of the coupling reaction in a mixture with unreacted (uncoupled) fatty chains.

It should be observed that the reaction is preferably performed in water and in case the acid is not water-soluble, the acid is first solved in a water soluble solvent like a glycol, in particular butylene glycol.

Said complexes constitute the first object of the present invention. The compositions, notably cosmetic, pharmaceutical or food compositions, containing them in a compatible excipient constitute the second object of the said invention.

Said compositions generally contain from 0.01 to 40% by weight of such complex(es) and advantageously from 0.1 to 10% by weight.

More specifically, the notably cosmetic, pharmaceutical or food compositions which contain at least one protein as active ingredient are an integral part of the present invention; said protein which intervenes, being at least in part (even in totality) as a complex such as described above. For the elaboration of said compositions, said complex may be used purified (isolated from the reaction mixture in which it was synthesised) or as a mixture with one and/or the other(s) of the reagents which intervened in its synthesis. According to this second variant, the reaction mixture (at the end of the reaction) is advantageously used which contains said complex and the unreacted reagents (principally fatty chains insofar as they intervene in excess).

The compositions wherein said complexes intervene as emulsifying agents also make a part of the invention.

In any case, it was noted in a surprising way that the complexes of the invention have hydrating and emulsifying properties. This is relatively unexpected insofar as the person skilled in the art cannot ignore that the proteins have a capacity to trap water which is much lower than that of polysaccharides and insofar as said capacity, which is relatively low in the absolute, should have been affected by the lipophilisation of said proteins.

In addition to these hydrating and emulsifying properties, which are relatively unexpected, the complexes of the invention have revealed to have other properties which are totally unexpected.

It is as such that a soluble wheat protein having an average molecular mass of 100,000 Daltons, onto which fatty chain (s) has been grafted, notably stearic, palmitic, phtaloyl, terephtaloyl, sebacoyl, glutaryl, adipoyl or succinyl acid chains, and mixtures thereof have been grafted, have extremely strong skin restructuration properties, which enable one to envisage the use of this lipophilised protein (complex in the sense of the invention) in applications wherein a destructuration of the epidermis is observed (physico-chemical damaging effects or skin ageing . . . ).

Similarly, a soluble almond protein having an average molecular mass of 30,000 Daltons, onto which fatty chain (s) has been grafted, notably stearic, palmitic, phtaloyl, terephtaloyl, sebacoyl, glutaryl, adipoyl or succinyl acid chains, and mixtures thereof have been grafted, has the property of calming moderate to strong sunburn, which enables envisaging the use of this lipophilised protein (complex in the sense of the invention) in sun or after-sun formulations.

Similarly, an insoluble laminary protein having an average molecular mass of 10,000 Daltons onto which fatty chain (s) has been grafted, notably caprylic, stearic, palmitic, phtaloyl, terephtaloyl, sebacoyl, glutaryl, adipoyl or succinyl acid chains, and mixtures thereof, have been grafted has the property of inhibiting a certain number of micro-organisms, which enables one to envisage the use of this lipophilised protein (complex in the sense of the invention) in applications wherein the destruction of micro-organisms is envisaged (anti-acne effects, anti-dandruff effects, anti-body odour effects, natural preservative . . . ).

It has previously been insisted upon the diversity of the complexes of the invention. The interest of such a diversity is herein referred to.

Furthermore, it is recalled herein that the properties of the complexes of the invention, whether they are more or less unexpected, are expressed as expected, on the Stratum corneum, by their lipophilisation.

The compositions of the invention therefore consist essentially of cosmetic compositions. The invention further encompasses therapeutic, food or dietary compositions which are particularly efficient on the mucous membranes.

According to its third aspect, the invention relates to a method of preparing amphiphilic (hydrolipophilic) complexes described above. Said method characteristically comprises the reaction, at a temperature between ambient temperature and 80° C., preferably in aqueous medium or exceptionally in a solvent medium, between at least one protein or polypeptide of the aforementioned type and at least one fatty chain of the aforementioned type, said reagents intervening in a [protein or polypeptide/fatty chain] in a weight ratio between 1000/1 and 1/10 and advantageously with the first and second embodiments ratios above described. Said reaction may be qualified a grafting reaction or more exactly a coupling reaction (insofar as it does not generate only covalent bonds between the reagents).

Said reaction is carried out at a relatively low temperature. The degradation of the reactive proteins is thus minimised. The reaction brings in or does not bring in a solvent, preferably a aqueous medium, or exceptionally a organic solvent, notably when the acid is not water-soluble and in such a case the solvent is advantageously a water-soluble glycol, in particular butylene glycol. The intervention of such a solvent may be done away with if, at the temperature of the reaction, the reagents are liquid.

At the end of the reaction, the “binary” complexes are generally not isolated. They are found thus in a mixture principally with the unreacted fatty chains.

It is generally desired to adjust the pH of the complexes obtained in order to render it compatible with the later, notably cosmetic applications. The pH is adjusted to values between 2 and 10 and more particularly between 5 and 7. To this end, neutralising agents are used which are selected from:

inorganic bases (such as KOH, NaOH, Ca(OH)₂. . . );

metal bases (as hydroxide, carbonate . . . );

organic bases (citrate, phosphate, borate, acetate, TRIS . . . buffers; C₁-C₆ amines or alkylamines: triethanolamine, aminomethylpropane . . . ).

After preparing the complexes whose pH, if need was, adjusted to pHs compatible with their later use (said pH is advantageously adjusted by dispersion of said complexes in the aqueous phase), it is possible to dry them by atomisation, lyophilisation, dehydration under vacuum . . . Said dried complexes, or directly obtained without water, may then be made into a form, notably in the form of turnings.

The coupling reaction carried out may be carried out chemically or enzymatically. The enzymatic route is, within the context of the present invention, totally original.

According to said chemical route, it is possible to:

Either react the fatty chains—fatty acids, fatty alcohols, fatty amines—under conventional conditions of peptide synthesis; i. e. in the presence of bifunctional agents, such as diimides;

or react the fatty acids in reactive (more reactive) forms i.e. react halides (chlorides, bromides, iodides . . . ) of fatty acids, fatty acid anhydrides, fatty acid anhydride derivatives, as is well known to one skilled in the art.

According to said original enzymatic route, the proteins are coupled to the fatty chains in the presence of an enzyme, generally at a temperature between 30 and 70° C. Advantageously said temperature is between 50 and 60° C. Advantageously, the intervening enzyme is an acyltransferase. According to three variants of this enzymatic route, said enzyme is a lipase, notably selected from Mucor miehei lipase, pig pancreas lipase, Rhizopus arrhizus lipase, Candida lipase, Bacillus lipase and Aspergillus lipase, or a protease, notably consisting of papaine, or an amidase. Such an enzymatic reaction ensures, as the chemical reactions recalled above, the grafting of fatty chains onto the proteins. Said fatty chains, when they are fatty acids, can intervene as esters (including esters of glycerides). The enzyme present in the reaction medium ensures, firstly, the trans-esterification.

The reaction carried out, chemical or enzymatic, ensures the coupling by generating ester and/or amide covalent bonds. It has been seen that said coupling also brings in ionic bonds, hydrogen bonds, Van der Waals type forces.

The reaction is advantageously carried out with a water activity of the reaction medium (aw) between 0.2 and 1 and advantageously between 0.3 and 0.7.

The methods of preparing compositions of the invention, notably 10 cosmetic, pharmaceutical and food compositions containing amphiphilic complexes also make up a part of the invention. They principally consist in admixing the active ingredient with an appropriate excipient. It has been seen that said active principle could have emulsifying properties. This can reveal to be particularly interesting. The intervention of any synthetic emulsifying agent may thus be limited, even eliminated.

The invention is illustrated, under its various aspects, by the Examples below.

In the specification all the percentages indicated are by weight, the temperature is the ambient temperature or expressed in Degrees Celsius, and the pressure is the atmospheric pressure, unless otherwise indicated.

### EXAMPLE 1

Preparation of a Lauric Acid (C12)—Soya Protein Complex.

1,660 g of lauric acid of purity equal to 99% are heated to 60° C. in a reactor under nitrogen. After fusion of the lauric acid chains and obtaining a colourless oil, 470 g of a soya isolate (average molecular weight: 50,000 D), containing at least 96% of native proteins, are then added in a fine stream into the reactor under moderate mechanical stirring.

After a homogeneous suspension is obtained, 300 g of lipase extracted from Mucor miehei, immobilised on macroporous ion exchange resin (commercial name: Lipozyme® from Novo) are added to the reactor. The whole is kept for 15 days at 600 C in a closed reactor under moderate mechanical stirring.

After 15 days' reaction, the complex is filtered at 90° C. so as to remove the enzyme. The complex thus obtained is made into turnings during its cooling.

After analysis, it proves to be that this complex is constituted of lipophilised proteins of which about 16% of the free amine functions (lateral and terminal) were grafted by the fatty acids (lauric acid).

This complex is a beige powder of turnings of characteristic odour. It may be used in a cosmetic formulation at 3% and, by virtue of the amphiphilicity brought about by the grafting, it is possible to incorporate it in the aqueous and/or oily phases of a cosmetic/dermocosmetic, pharmaceutical/dermopharmaceutical, food/dietary.

### EXAMPLE 2

Preparation of a Stearic (Ci8) Acid and Palmitic (C16) Acid—Soya Protein Complex.

270 g of stearic acid and 180 g of palmitic acid, each of greater than 90% purity, are placed in 1,000 ml of tert-butanol, and then heated to 60° C. in a reactor under nitrogen. After fusion of the acid chains and obtaining a colourless oil, 150 g of an soya isolate (average molecular mass: 50,000 D), which contains at least 96% of native proteins, are then added in a fine stream into the reactor under moderate mechanical stirring.

After a homogeneous suspension is obtained, 45 g of lipase extracted from Rhizopus arrhizus are added to the reactor. The whole us kept for 21 days at 55° C. in a closed reactor under moderate mechanical stirring.

After 21 days' reaction, the complex is heated at 90° C. for 20 minutes so as to inactivate any residual enzymatic activity. The tert-butanol is then removed by distillation under reduced pressure. The complex thus obtained is made into turnings during its cooling.

After analyses, it proves to be that the complex is constituted of lipophilised proteins of which about 21% of the lateral amine functions were grafted by the fatty acids.

This complex is a beige powder of turnings of characteristic odour. It may be used in a cosmetic formulation at 3% and, by virtue of the amphiphilicity brought about by the grafting, it is possible to incorporate it in the aqueous and/or oily phases of a cosmetic/dermocosmetic, pharmaceutical/dermopharmaceutical, food/dietary, composition.

### EXAMPLE 3

Preparation of a Stearic(C18) and Palmitic (C16) Acid—Wheat Protein Complex.

Carried out as described in Example 2 by substituting the 150 g of the soya isolate with 150 g of an atomisate obtained from a solution of wheat protein (average molecular mass: 100,000 D). A complex of the type described in Example 2 is obtained (beige coloured turnings of characteristic odour).

### EXAMPLE 4

Preparation of a Stearic (Cl 8) Acid and Palmitic (Cl 6) Acid—Almond Protein Complex.

300 g of stearic acid and 140 g of palmitic acid, each of greater than 90% purity, are placed in 1,000 ml of isopropanol, and then heated to 60° C. in a reactor under nitrogen. After fusion of the acid chains and obtaining a homogeneous oily phase, 150 g of an lyophilisate obtained from a solution of almond protein (average molecular mass: 30,000 D), are then added in a fine stream into the reactor under moderate mechanical stirring.

After a homogeneous suspension is obtained, 35 g of lipase extracted from Rhizopus arrhizus are added to the reactor. The whole is kept for 12 days at 55° C. in a closed reactor under moderate mechanical stirring.

After 12 days' reaction, the complex is heated at 90° C. for 20 minutes so as to inactivate any residual enzymatic activity. The isopropanol is then removed by distillation under reduced pressure. The complex thus obtained is made into turnings during its cooling.

This complex is a beige powder of turnings of characteristic odour. It may be used in a cosmetic formulation at 3% and, by virtue of the amphiphilicity brought about by the grafting, it is also possible to incorporate it in the aqueous and/or oily phases of a cosmetic/dermocosmetic, pharmaceutical/dermopharmaceutical, food/dietary, composition.

### EXAMPLE 5

Carried out as described in Examples 2 to 4 but the intervening solvent is selected from hexane, chloroform, cyclohexane, chloromethane, dichloromethane, trichloromethane, diethyl ether, methyl tert-butyl ether, or a mixture of these solvents.

### EXAMPLE 6

Carried out as described in the preceding examples but in varying the nature of the enzyme used: Mucor miehei lipase, pig pancreas lipase, Rhizopus arrhizus lipase, Candida lipase, Bacillus lipase, Aspergillus lipase, or other acyltransferases.

### EXAMPLE 7

Carried out as described in the preceding examples but in varying the nature of the intervening protein: wheat protein, oat protein, maize protein, almond protein, soya protein.

### EXAMPLE 8

Carried out as described in the preceding examples but in varying the parameters of the coupling reaction below:

the proportion between fatty chains and proteins (or polypeptides);

the reaction temperature (between ambient temperature and 80° C.);

reaction time (from 30 minutes to 21 days).

### EXAMPLE 9

Carried out as described in the preceding examples but in varying the nature of the intervening fatty acid: heptanoic (C7) acid, octanoic (C8) acid, decanoic (C10) acid, lauric (C12) acid, myristic (C14) acid, palmitic (C16) acid, stearic (C18) acid, ricinoleic (C18) acid, oleic (C18) acid, linoleic (C18) acid, linolenic (C18) acid, other fatty acids with shorter or longer chain saturated, unsaturated or polyunsaturated fatty acids, used pure or as a mixture.

### EXAMPLE 10

Carried out as described in the preceding examples but the fatty acids which are reacted with the proteins are in the form of esters, and the lipase used effects a transesterification and/or transacylation reaction. Thus, ethyl linoleate, isopropyl oleate and glycerol linoleate as well as various vegetable oils in the form of triglycerides (of which coconut oil) were used to provide the fatty chain which will then come to graft onto the protein.

### EXAMPLE 11

Carried out as described in Examples 1 to 9 but the fatty acids which are reacted with the proteins are in a reactive form, of the type acid halide or acid anhydride. In this case, the reaction can be carried out in water at ambient temperature and does not necessitate lipase. Such reactions are explicitly described in Examples 17 and 18.

### EXAMPLE 12

Carried out as described in examples 1 to 8 but the fatty chains which are reacted with the proteins are in the alcohol form (formation of ester bonds with the carboxylic acid functions of the protein) or in the amine form (formation of amide bonds with the carboxylic acid functions of the protein). Such reactions are explicitly described in examples 14 and 16 below.

### EXAMPLE 13

Carried out as described in examples 2 to 12 but without using solvent.

### EXAMPLE 14

Preparation of a Decyl Alcohol (C10)—Soya Protein Complex

300 g of decyl alcohol, of greater than 90% purity, are heated to 60° C. in the presence of 670 ml of tert-butanol in a reactor under nitrogen. After fusion of the decyl alcohol chains and obtaining a colourless oil, 100 g of a soya isolate (average molecular mass: 50,000 D), containing at least 96% of native proteins, are then added in a fine stream into the reactor under moderate mechanical stirring.

After a homogeneous suspension is obtained, 30 g of lipase extracted from Rhizopus arrhizus are added to the reactor. The whole is kept for 10 days at 600 C in a closed reactor under moderate mechanical stirring.

After 10 days' reaction, the complex is heated at 90° C. for 20 minutes 50 as to inactivate any residual enzymatic activity. The tert-butanol is then removed by distillation under reduced pressure. The complex thus obtained is made into turnings during its cooling.

After analysis, it proves to be that this complex is constituted of proteins lipophilised with fatty chains, and about 12% of the fatty alcohols were coupled to the protein (with the aid of covalent bonds such as ester functions, but also with the aid of ionic functions).

This complex is a beige powder of turnings of characteristic odour. It may be used in a cosmetic formulation at 3% and, by virtue of the amphiphilicity brought about by the grafting, it is possible to incorporate it in the aqueous and/or oily phases of a cosmetic/dermocosmetic, pharmaceutical/dermopharmaceutical, food/dietary, composition.

### EXAMPLE 15

Carried out as described in the preceding examples but the complexes formed are dispersed in the aqueous phase and their pH is adjusted so as to be compatible with cosmetic formulations, with the aid of an inorganic or organic base. The complexes thus obtained can then be dried by atomisation, lyophilisation or drying under vacuum.

### EXAMPLE 16

Preparation of a Laurylamine (C12)—.Soya Protein Cornplex.

300 g of laurylamine, of greater than 90% purity, are heated to 60° C, in the presence of 670 ml of tert-butanol in a reactor under nitrogen. After fusion of the laurylamine chains and obtaining a colourless oil, 100 g of a soya isolate (average molecular mass: 50,000 D), containing at least 96% of native proteins, are then added in a fine stream into the reactor under moderate mechanical stirring.

After a homogeneous suspension is obtained, 30 g of lipase extracted from Rhizopus arrhizus are added to the reactor. The whole is kept for 10 days at 60° C. in a closed reactor under moderate mechanical stirring.

After 10 days' reaction, the complex is heated at 90° C. for 20 minutes so as to inactivate any residual enzymatic activity. The tert-butanol is then removed by distillation under reduced pressure. 4,000 ml of water are then added to the complex and the whole is brought up to 70° C. under moderate stirring; then, about 1.5 moles of HC1 (as a 6N solution) are added slowly to the reaction mixture so as to obtain a pH between 5.0 and 7.0. The complex thus obtained is then dried by lyophilisation.

After analysis, it proves to be that this complex is constituted of proteins lipophilised with fatty chains, and about 11% of the fatty amines were coupled to the protein (with the aid of covalent bonds such as ester functions, but also with the aid of ionic functions).

This complex is a beige powder of turnings of characteristic odour. It 20 may be used in a cosmetic formulation at 3% and, by virtue of the amphiphilicity brought about by the grafting, it is possible to incorporate it in the aqueous and/or oily phases of a cosmetic/dermocosmetic, pharmaceutical/dermopharmaceutical, food/dietary, composition.

### EXAMPLE 17

Preparation of a Stearic (Cl 8) and Palmitic (C16) Acids—Wheat Protein Cornplex.

100 g of soluble wheat protein of high average molecular mass (100,000 D) extracted from wheat gluten are placed in 5,000 ml of demineralised water. The reaction mixture is adjusted to pH 11 with a sodium hydroxide solution (NaOH, 12N). Under very strong stirring of the Ultraturrax or Silverson type (10,000 to 20,000 rpm), 300 g of a mixture of stearic and palmitic acid chlorides are then added slowly. The pH goes in a few tens of minutes from 11 to neighbouring 1 when a buffer is not added to the reaction mixture. After a reaction time of about one hour at ambient temperature, the whole is neutralised to a pH neighbouring 7.0 with a sodium hydroxide solution (NaOH, 12N). The whole is then lyophilised and then optionally sterilised by gamma or beta rays. The product is a white pulverulent powder which can be placed in both aqueous phases and oily phases of the cosmetic preparations for example. Part of the fatty acids has reacted with the protein to form amide and ester bonds, and a part has not reacted finds itself nevertheless strongly complexed by hydrogen bonds and by Van der Waals forces to the protein.

### EXAMPLE 18

Preparation of a Stearic (C18) and Palmitic (C16) Acids—Almond Protein Complex.

The same technique of grafting is carried out as in Example 17 above, but an almond protein, of average molecular mass near to 30,000 D is used instead of the wheat protein. The reaction is carried out in regulating the pH to il by adding sodium carbonate. The complex leaving this grafting is kept in the liquid form and contains 5% dry matter; 0.2% parabens and 0.5% xanthane gum are then added. The complex thus formed is marketed under the form of this solution thus described.

### EXAMPLE 19

Carried out as described in examples 17 and 18 but in operating at temperatures between 20 and 100° C. The grafting reactions carried out at very high temperatures give better yields but give rise to moderate to severe degradations of the proteins used.

### EXAMPLE 20

Preparation of a Hexadecylamine (Cl 6)—Oat Protein Complex

80 g of oat protein of average molecular mass equal to 6,000 D are placed in 4,000 mi of demineralised water. The medium is neutralised to pH 7.0. A sufficient quantity of phosphate buffer is added so as to obtain a 0.5M phosphate buffer in the reaction medium i mole of a carbodiimide such as for example 190 g of N-(dimethylamino-3-propyl)-N′-ethylcarbodiimide hydrochloride is then added to the mixture under stirring; 240 g of 1-hexadecylamine, beforehand placed in suspension in 2,000 ml of water brought to 80° C., are then added to the reaction mixture under very powerful mechanical stirring (Ultraturrax type, 10,000 to 20,000 rpm). The whole is kept under stirring for 1 hour at ambient temperature or 24 hours at 6° C., and then adjusted to a pH of 7.0. It is then optionally dialysed against distilled water for 48 hours at 6° C., optionally dried by lyophilisation, and then optionally sterilised with beta or gamma rays.

The covalent bond which results from this reaction provides the amide bonds, but other characteristic bonds are present between the fatty amine and the polypeptide, such as ionic bonds and Van der Waals type forces.

### EXAMPLE 21

Preparation of a Dihalide or Dichloride Acid—Wheat Protein Complex

100 g of dry soluble wheat protein of high average molecular mass (100,000 D) extracted from wheat gluten are placed in 5,000 ml of demineralised water. The ratio mixture is adjusted to pH 11 with a sodium hydroxyde solution (NaoH, 12N), under very strong stirring of the ultraturrax or Silverson type (10,000 to 20,000 rpm). 300 mg of a dihalide acid selected from the group comprising:

Ex 21 A: phtaloyl dihalide (preferred dichloride),

Ex 21 B: terephtaloyl dihalide (preferred dichloride),

Ex 21 C: sebacoyl dihalide (preferred dichloride),

EX 21 D: glutaryl dihalide (preferred dichloride),

EX 21 E: adipoyl dihalide (preferred dichloride),

Ex 21 F: succinyl dihalide (preferred dichloride),

are then added slowly. The pH goes in a few tens of minutes from 11 to neighbouring 1 when a buffer is not added to the reaction mixture.

The weight ratio expressed as g/g between protein and dihalide is 1000:3 in this case.

After a reaction time of about one hour at ambient temperature, the whole is neutralised to a pH neighbouring 7.0 with a sodium hydroxyde solution (NaoH 12N).

The whole can be optionally lyophilised and then optionally sterilised by gamma or beta rays.

The dihalide acid preferred in this example is sebacoyl chloride of Ex 21C.

21 G: The ratio between protein and dihalide could be also adjusted to 1000 1 in that case to 100 g of protein is added 100 mg of dihalide.

21 H: The ratio between protein and dihalide could be also adjusted to 1:3 in that case to 100 g of protein is added 300 g of dihalide.

21 I: Dihalide could be replaced by polyhalide, preferaly trihalide, triacid chlorides, for example citrate trichloride.

21 J : Dihalide could be replaced by di-anhydrides (succinyl anhydride, malyl anhydride etc . . . )

21 K: pH could be adjusted with strong bases such as NaOH, KOH, but also with weak bases such as buffering agents (phosphate, succinate, citrate . . . ).

### EXAMPLE 22

Preparation of a Dihalide or Dichloride Acid—Almond Protein Complex

The same technique of crosslinking is carried out as in example 21 above, but an almond protein, of average molecular mass to 30,000 D is used instead of the wheat protein.

### EXAMPLE 23

Preparation of a Dihalide or Dichloride Acid—Lupin Protein Complex

The same technique of crosslinking is carried out as in example 21 above, but an lupin protein, (genus Lupinus) is used instead of the wheat protein.

### EXAMPLE 24

Preparation of a Dihalide or Dichloride Acid—Soya Protein Complex

The same technique of crosslinking is carried out as in example 21 above, but an soya protein, (genus Glycine) is used instead of the wheat protein.

### EXAMPLE 25

Preparation of a Dihalide or Dichloride Acid—Pea Protein Complex

The same technique of crosslinking is carried out as in example 21 above, but an pea protein, (genus Pisum) is used instead of the wheat protein.

### EXAMPLE 26

Preparation of a Dihalide or Dichloride Acid—Chick Pea Protein Complex

The same technique of crosslinking is carried out as in example 21 above, but an chick pea protein, (Cicer) is used instead of the wheat protein.

### EXAMPLE 27

Preparation of a Dihalide or Dichloride Acid—Lucerne Protein Complex The same technique of crosslinking is carried out as in example 21 above, but an lucerne protein (Medicago), is used instead of the wheat protein.

### EXAMPLE 28

Preparation of a Dihalide or Dichloride Acid—Horse Bean Protein Complex

The same technique of crosslinking is carried out as in example 21 above, but an horse bean protein (Vicia), is used instead of the wheat protein.

### EXAMPLE 29

Preparation of a Dihalide or Dichloride Acid—Lentil Protein Complex

The same technique of crosslinking is carried out as in example 21 above, but an lentil protein, (Lens) is used instead of the wheat protein.

### EXAMPLE 30

Preparation of a Dihalide or Dichloride Acid—Bean Protein Complex

The same technique of crosslinking is carried out as in example 1 above, but an bean protein, (Phaseolus) is used instead of the wheat protein.

### EXAMPLE 31

Preparation of a Dihalide or Dichloride Acid—Colza Protein Complex

The same technique of crosslinking is carried out as in example 21 above, but an colza protein, (Brassica) is used instead of the wheat protein.

### EXAMPLE 32

Preparation of a Dihalide or Dichloride Acid—Sunflower Protein Complex

The same technique of crosslinking is carried out as in example 21 above, but an sunflower protein, (Helianthus) is used instead of the wheat protein.

### EXAMPLE 33

Preparation of a Dihalide or Dichloride Acid—Maize Protein Complex

The same technique of crosslinking is carried out as in example 21 above, but an maize protein, is used instead of the wheat protein.

### EXAMPLE 34

Preparation of a Dihalide or Dichloride Acid—Barley Protein Complex

The same technique of crosslinking is carried out as in example 21 above, but an barley protein, is used instead of the wheat protein.

### EXAMPLE 35

Preparation of a Dihalide or Dichloride Acid—Malt Protein Complex

The same technique of crosslinking is carried out as in example 21 above, but a malt protein, is used instead of the wheat protein.

### EXAMPLE 36

Preparation of a Dihalide or Dichloride Acid—Oats Protein Complex

The same technique of crosslinking is carried out as in example 21 above, but an oats protein, is used instead of the wheat protein.

### EXAMPLE 37

Tolerance and Toxicity:

5 km and ocular irritation studies (carried out according to the protocols in accordance with the OCDE directives No. 404 (12 May 1981) and No. 405 (24 Feb. 1987)), were carried out with several of the products obtained according to the examples above (Examples 1 to 36) in the form of solutions at 10%. In every case, the products appeared as being “non-iritant” (did not provoke any sign of skin or ocular irritation), and were extremely well tolerated.

Similarly, the administration by the oral route of maximal doses of 5 g of these products per kilogram of body weight did not provoke any toxicity (tests carried out according to a protocol in accordance with the directive une of the OCDE relating to the study of the toxicity by the oral route (No. 401 (24 Feb. 1987)).

Furthermore, sensitisation tests according to the protocol of Magnusson and Kligman were carried out with these products in solution in water at 10% and these products were classed amongst the products not having a sensitising property.

### EXAMPLE 38

Anti-age. restructuring formulation CC591:

| | | | Quantities |
|---|---|---|---|
| Phase | Products | INCI names | (%) |
| A | Brij 72 | Steareth 2 | 3 |
| | Brij 721 | Steareth 21 | 2 |
| | Isostearyl Isostearate | Isostearyl | 4 |
| | | Isostearate | |
| | Apricot kernel oil | Apricot Kernel Oil | 4 |
| | Huile de safran | Safflower Oil | 2 |
| | Dimethicone 556 | Dimethicone 556 | 2 |
| | Crodacol CS50 | Ketostearyl Alcohol | 3 |
| B | Water | Water | qs for 100 |
| | Glycerine | Glycerine | 5 |
| | Product of the invention according to any | | 6 |
| | one of examples 1 to 36, preferably | | |
| | example 2 | | |
| C | Phenonip ® | Phenoxyethanol | 0.5 |
| | | Methylparaben | |
| | | Ethylparaben | |
| | | Propylparaben | |
| | | Butylparaben | |
| D | Propylene glycol | Propylene Glycol | 0.5 |
| | Perfume | | 0.3 |
| | Alpha tocopherol | Alpha Tocopherol | 0.05 |

Phases A and B are heated separately under moderate stirring. The pH of phase B is adjusted to the desired value. A is poured into B under very vigorous stirring (Silverson or Ultraturrax type), the temperature is then allowed to drop under slow stirring. At 30° C., the components of phases C and D are added.

### EXAMPLE 39

Anti-age face formulation

| | | | Quantities |
|---|---|---|---|
| Phase | Products | INCI names | (%) |
| A | Isostearyl Isostearate | Isostearyl Isostearate | 4 |
| | Huile de Carthame | Safflower Oil | 4 |
| | Cetiol J600 | Oleyl Erucate | 2 |
| | Dimethicone 556 | Dimethicone | 5 |
| | Crodacol CS50 | Ketostearyl Alcohol | 3 |
| | Product of the invention according to any | | 3 |
| | one of examples 1 to 36, preferably | | |
| | example 17 | | |
| B | Glycerine | Glycerine | 5 |
| | Water | Water | qs for 100 |
| C | Phenonip ® | Phenoxyethanol | 0.5 |
| | | Methylparaben | |
| | | Ethylparaben | |
| | | Propylparaben | |
| | | Butylparaben | |
| | Propylene glycol | Propylene Glycol | 0.5 |
| D | Perfume | Perfume | 0.3 |

Phases A and B are heated separately under moderate stirring. The pH of the formula is conditioned in this case by the pH of the product of the invention. A is poured into B under very vigorous stirring (Silverson or Ultraturrax type), the temperature is then allowed to drop under slow stirring. At 30° C., the components of phases C and D are added. If need be, the preparation is adjusted to the desired pH with the aid of lactic acid for example.

### EXAMPLE 40

Dry skin. face formulation

| | | | Quantities |
|---|---|---|---|
| Phase | Products | INCI names | (%) |
| A | Huile de bourrache | Borrage Oil | 2 |
| | Huile de Carthame | Safflower Oil | 4 |
| | Myritol 318 | Caprylic/Capric | 6 |
| | Crodacol CS50 | triglyceride | 3 |
| | | Ketostearyl Alcohol | |
| B | Glycerine | Glycerine | 5 |
| | Water | Water | qs for 100 |
| | Product of the invention according | | 4 |
| | to any one of examples 1 to 36, | | |
| | preferably example 2 | | |
| C | Phenonip ® | Phenoxyethanol | 0.5 |
| | | Methylparaben | |
| | | Ethylparaben | |
| | | Propylparaben | |
| | | Butylparaben | |
| | Propylene glycol | Propylene Glycol | 0.5 |
| D | Perfume | Perfume | 0.3 |

Phases A and B are heated separately under moderate stirring. The pH of phase B is adjusted to the pH of the formulation desired. A is poured into B under very vigorous stirring (Silverson or Ultraturrax type), the temperature is then allowed to drop under slow stirring. At 30° C., the components of phases C and D are added. If need be, the preparation is adjusted to the desired pH with the aid of lactic acid for example.

### EXAMPLE 41

Family shampoo formulation

| | | | Quantities |
|---|---|---|---|
| Phase | Products | INCI names | (%) |
| A | Texapon N40 ® (Henkel) | sodium laureth | 40 |
| | Comperlan KD ® | sulphate | 2 |
| | (Henkel) | Cocamide DEA | |
| B | Product of the invention according to any one | | 0.3 |
| | of examples 1 to 36, preferably example 17 | | |
| | Water | Water | qs for 100 |
| | sodium chloride | sodium chloride | 1.5 |
| C | Phenonip ® | Phenoxyethanol | 0.5 |
| | | Methylparaben | |
| | | Ethylparaben | |
| | | Propylparaben | |
| | | Butylparaben | |
| | Propylene glycol | Propylene Glycol | 0.5 |

Phase B is heated separately at 75° C. under moderate stirring. The pH of said phase B is adjusted to the pH of the formulation desired. B is poured into A at 20° C. under very slow stirring, the temperature is then allowed to drop. At 30° C., phase C is added.

### EXAMPLE 42

Mild shampoo formulation

| | | | quantities |
|---|---|---|---|
| Phase | Products | INCI names | (%) |
| A | Tween 20 ® (ICI) | Polysorbate 20 | 10 |
| | TegoBetaine L7 ® | Cocamidopropyl | 10 |
| | (Goldschmidt) | Betaine | 3 |
| | Atlas G1821 ® (ICI) | PEG-150 Distearate | |
| B | Product of the invention according to any one of | | 0.5 |
| | examples 1 to 36, preferably example 18 | | |
| | Water | Water | qs for 100 |
| C | Phenonip ® | Phenoxyethanol | 0.5 |
| | | Methylparaben | |
| | | Ethylparaben | |
| | | Propylparaben | |
| | | Butylparaben | |
| | Propylene glycol | Propylene Glycol | 0.5 |

Phase B is heated separately at 75° C. under moderate stirring. The pH of said phase B is adjusted to pH of the formulation desired. A is homogenised at 20° C. B is poured into A at 20° C. under very slow stirring, the temperature is then allowed to drop. At 30° C., phase C is added.

### EXAMPLE 43

Pearlescent shampoo formulation:

| | | | Quantities |
|---|---|---|---|
| Phase | Products | INCI names | (%) |
| A | Texapon N40 ® (Henkel) | Sodium Laureth Sulphate | 40 |
| | Comperlan KD ® (Henkel) | Cocamide DEA | 2 |
| | Euperlan PK771 ® | Glycol Distearate (and) | 4 |
| | (Henkel) | Sodium Laureth Sulphate | |
| | | (and) Cocamide MEA | |
| | | (and) Laureth-10 | |
| B | Product of the invention according to any one of | | 0.5 |
| | examples 1 to 36, preferably example 16 | | |
| | Water | Water | qs for 100 |
| | Sodium chloride | Sodium Chloride | 1.5 |
| C | Phenonip ® | Phenoxyethanol | 0.5 |
| | | Methylparaben | |
| | | Ethylparaben | |
| | | Propylparaben | |
| | | Butylparaben | |
| | Propylene glycol | Propylene Glycol | 0.5 |

Phase B is heated separately at 75° C. under moderate stirring. The pH of phase B is adjusted to the pH of the formulation desired. B is homogenised at 200 C. B is poured into A at 20° C. under very slow stirring and then the temperature is allowed to drop. At 30° C., phase C is added.

### EXAMPLE 44

Use of a Stearic and Palmitic Acids—Wheat Protein Complex in ‘^{t}Restructuring¹’ Cosmetic Applications and Which Enable Fighting Against Ageing Effects.

The complexes prepared out according to Examples 3 (enzymatically) and 17 (chemically) were tested for their capacity to smoothen the skin micro-relief. The outside appearance of the skin does in fact reveal its general state, and the meshes formed by the skin micro-depressionary network have a tendency to grow and to dig in during ageing. Other external factors can also contribute to this phenomenon, as for example the use of detergents. This disorganisation of the micro-relief is the sign of an alteration of the horny layer and of its natural protective barrier function. It gives a rough appearance and a coarse integument touch and then leads to a pronounced dehydration of it.

The restructuring activity of these complexes was studied after an important destruction of the micro-depressionary network obtained by a chemical damaging effect of the skin coating with the aid of an aqueous solution which contains 10% detergent (sodium lauryl sulphate). The tests were carried out on the external side of both hands of 10 volunteers. Each hand was washed 4 times a day for 30 seconds, at 1/2 hour intervals for 4 days with this detergent solution. One of the hands received at the end of these treatments, each day, a treatment carried out from a solution containing 3% of the complex prepared according to example 3 or 17 of the invention. Every day, the skin repairing was evaluated in comparison to the control zones, damaged and non-treated, by direct observation of the skin surface under the stereo-microscope, and by stripping studies. The effectiveness of the complex was compared to that of the wheat protein used for the preparation of said complex; the filmogenic and softening powers of the wheat protein being well-known.

The two products (protein and complexed protein) clearly and almost obviously increase the visual appearance of the horny surface; however, only the fatty acids-wheat protein complex according to the invention has an extremely significant restructuring power (neighbouring 90%), which does not limit the slowing down of the damaging effect due to the detergent, but which allows a regeneration of the whole of the integument. Thus, the treated skins are frequently in a better state after degradation and application of the aqueous solution of the complex prepared in Example 3 or 17, than before any treatment.

Thus, it is possible to affirm that the stearic and palmitic chains-wheat protein complex is an active regenerating cosmetic capable of equilibrating and harmonising the cohesion of the epidermis.

### EXAMPLE 45

Use of a Stearic and Palmitic Acids—Almond Protein Complex in Cosmetic applications which allow soothing skin damaging effects linked to sunburn.

The complexes prepared according to examples 4 (enzymatically) and 18 (chemically) were tested for their capacity to reduce and to calm sunburn; in fact, repeated sunburns favour an alteration of the biochemical mechanisms of the skin, by the destruction of the lipids of the ceil membranes, by the fragmentation of the biological macromolecules which are indispensable to the skin reparations, and on the other hand the acceleration of the skin ageing; it being possible for the conjunction of these two phenomena to be however translated by the appearance of skin cancer.

The anti-sunburn power of the stearic and palmitic acids-almond protein complex was studied in the guinea pig whose sunburn reaction is well correlated with that of man. The irradiations of the animals were carried out with the aid of two Philipps TL 40W/12 lamps emitting between 280 and 340 nm with a peak at 315 nm. Placed at 3% within an emulsion (see composition A below), the complex prepared according to example 4 or 18 was tested in comparison to a placebo emulsion (see composition C below) and in comparison to an emulsion containing the almond polypeptide used in the complex, in a non-complexed form (see composition B below). In each case, 0.25 mi of product was administered immediately after irradiation, then 2, 5and 24 hours after exposure. The sunburn being evaluated according to a visual quotation of O (no sunburn) to 4 (intense sunburn), the sunburn reducing effect was measured 2, 5, 24 and 48 hours after irradiation, in comparison to the irradiated but non-treated control areas. The results were then expressed in percentage inhibition of the sunburn.

| Phase | Ingredients | A | B | C |
|---|---|---|---|---|
| A | Complex described in example 4 or | 3 | 0 | 0 |
| | 18 | 0 | 0.75 | 0 |
| | Almond polypeptide | qs for | qs for | qs for |
| | Water | 100 | 100 | 100 |
| B | Apricot kernel oil | 5 | 5 | 5 |
| | Isostearyl isostearate | 5 | 5 | 5 |
| | Oleyl erucate | 2 | 2 | 2 |
| | Ketostearylic alcohol | 3 | 3 | 3 |
| C | Silicone oil | 2 | 2 | 2 |
| | Parabens | 0.2 | 0.2 | 0.2 |

Preparation of the compositions : Phases A and B are heated separately at 75° C. After a good homogenisation, B is poured into A under very vigorous stirring, the whole is then allowed to cool under slow stirring. At 30° C., phase C is then added. Results : Moderate sunburns were effected on guinea pigs. They correspond to sunburns of index 1.5 at 24 hours. The soothing effect procured by the application of a prior art cosmetic emulsion (placebo preparation C) on the sunburns, although sensitive, remains insufficient to efficiently to combat the development of the inflammatory reaction. On the contrary, the anti-sunburn power of the complex (preparation A) us immediate (inhibition of the sunburn of about 30%, 2 hours after the application) and durable throughout the treatments (inhibition of 45%, 45% and 65% after 5, 24 and 48 hours respectively). The preparation carried out with the non-complexed almond polypeptide (preparation B) does not allow either obtaining such results.

Other results were obtained on much more pronounced provoked sunburns (sunburns index 2 at 24 hours). In this case, the efficiency of preparation A is much more pronounced compared to the efficiency obtained with other preparations.

It is therefore possible to affirm that the stearic and palmitic chains-almond protein complex used in a cosmetic formulation is an active ingredient which allows durably repairing the destructive effects of moderate even strong sunburns observed during prolonged exposures to the Sun.

The invention covers any technical feature which appears to be new from the specification taken as a whole, including the examples which form an integral part of the invention, over any prior art become known at any time. The invention also covers any technical equivalent to any technical feature claimed, by equivalent is meant a technical feature structurally different which has substantially the same function and provides a result of the same nature as a technical feature claimed.

What is claimed is:

1. An amphiphilic complex resulting from the reaction, at a temperature between ambient temperature and 80° C., between:
at least one protein or polypeptide whose average molecular mass is greater than or equal to 5,000 Daltons; and
at least one fatty chain containing component whose carbon atom number is between 4 and 30, selected from fatty acids, fatty alcohols, fatty amines and derivatives thereof with the exception of undecylenic acid when the fatty acid is in a weight ratio in excess to the protein,
the protein or polypeptide/fatty component weight ratio ranging from 1000/1 to 1/10.

2. The complex of claim 1, wherein said fatty chain containing component comprises a mono reactive function selected from a monoacid, a monoalcohol, a monoamine and mixtures thereof.

3. The complex of claim 2, wherein the protein or polypeptide/fatty component weight ratio is ranging from 1/1 to 1/10.

4. The complex of claim 1, wherein said fatty chain containing component is polyfunctional and comprises at least two grafting functional groups selected from acid or anhydrid, alcohol, amine, and any mixture thereof.

5. The complex of claim 1, wherein the protein or polypeptide/fatty component weight ratio is ranging from 1000/1 to 1/3.

6. The complex of claim 1, wherein the protein or polypeptide/fatty component weight ratio is ranging from 1000/1 to 10/1.

7. The complex of claim 1, wherein said polyfunctional fatty chain containing component is selected from a phtaloyl, terephtaloyl, sebacoyl, glutaryl, adipoyl and succinyl acid under reactive form selected from a dihalide and an anhydride.

8. The complex of claim 1, wherein the protein has an average molecular mass between 10,000 and 1,000,000 Daltons.

9. The complex according to claim 1, wherein the fatty chain has a carbon atom number between 6 and 20.

10. The complex of claim 1, wherein the reaction product is in admixture with the unreacted fatty chains.

11. The Complex of claim 1 wherein the protein is an animal protein selected from collagen, gelatine, albumin, ovalbumin, elastin, reticulin, fibronectin, keratin, silk, laminin, desmosin, isodesrnosin, extra-cellular matrix proteoglycans, caseins, lactalbumin, lactoglobulins, and an enzyme.

12. The complex of claim 1, wherein the protein is a plant protein selected from a leguminous plant protein, a cereal protein, selected from the group consisting of: lupin (genus Lupinus), soya (genus Glycine), pea (genus Pisum), chick pea (Cicer), lucerne (Medicago), horse bean (Vicia). lentil (Lens), broad bean, bean (Phaseolus), colza (Brassica) and sunflower (Helianthus), moderated wheat, maize, cotton, almond, and any hydrolysate or polypeptide thereof.

13. The Complex of claim 1, wherein the fatty chain is selected from the group consisting of heptanoic, octanoic, decanoic, lauric, myristic, palmitic, stearic, ricinoleic, oleic, linoleic, linolenic fatty acid; the corresponding fatty alcohol and fatty amine; and mixtures thereof.

14. A composition selected from a cosmetic composition, a pharmaceutical composition and a food composition, containing as active ingredient at least one amphiphilic complex as defined in claim 1, in a compatible excipient.

15. A composition selected from a cosmetic composition, a pharmaceutical composition and a food composition, containing as active ingredient at least one amphiphilic complex as defined in claim 2, in a compatible excipient.

16. A composition selected from a cosmetic composition, a pharmaceutical composition and a food composition, containing as active ingredient at least one amphiphilic complex as defined in claim 4, in a compatible excipient.

17. A composition selected from a cosmetic composition, a pharmaceutical composition and a food composition, containing as active ingredient at least one amphiphilic complex comprising a plant protein from a leguminous plant selected from the group consisting of: lupin (genus Lupinus), soya (genus Glycine), pea (genus Pisum), chick pea (Cicer), lucerne (Medicago), horse bean (Vicia), lentil (Lens), bean (Phaseolus), colza (Brassica) and sunflower (Helianthus), in a compatible excipient.

18. A composition selected from a cosmetic composition, a pharmaceutical composition and a food composition, containing as active ingredient at least one amphiphilic complex comprising a plant protein from a cereal selected from the group consisting of wheat, maize, barley, colza, Lucerne, malt and oats, in a compatible excipient.

19. The composition of claim 18, wherein the plant protein is used in the form of a pulverulent preparation selected from the group consisting of a flour, a concentrate, an isolate, a liquid preparation, and a soya milk.

20. A method of preparing an amphiphilic complex comprising the reaction, at a temperature between ambient temperature and 80° C., between:
at least one protein or polypeptide whose average molecular mass is greater than or equal to 5,000 Daltons; and
at least one fatty chain containing component whose carbon atom number is between 4 and 30, selected from fatty acids, fatty alcohols, fatty amines and derivatives thereof with the exception of undecylenic acid when the fatty acid is in a weight ratio in excess to the protein, the protein or polypeptide/fatty component weight ratio ranging from 1000/1 to 1/10.

21. The Method of claim 20, wherein after said reaction, the complexes formed are dispersed in an aqueous phase for an adjustment of their pH, and are optionally dried afterwards.

22. The method of claim 20, wherein the protein or polypeptide and fatty chain are coupled chemically in the presence of bifunctional agents commonly used in peptide synthesis or by bringing in the fatty acids in a reactive form.

23. The method of claim 20, wherein the protein or polypeptide and fatty chain are coupled enzymatically; the fatty acids intervening optionally in the form of esters.

24. The method of claim 11, wherein said enzymatic coupling is carried out at a temperature between 30 and 70° C.

25. The method of claim 23, wherein said enzymatic coupling is carried out with an enzyme selected from the group consisting of an acyltransferase; a lipase, a Mucor miehei lipase, pig pancreas lipase, Rhizopus arrhizus lipase, Candida lipase, a Bacillus lipase, a Aspergillus lipase, a protease, papaine, and an amidase.

26. The method of claim 23, wherein the water activity (aw) of the reaction medium is between 0.2 and 1.

27. A method of cosmetic care comprising the topical delivery on a body area selected from the skin, scalp, hair and phanere, of a cosmetically effective amount of a cosmetic composition as defined in claim 14.

28. A method of cosmetic care comprising the topical delivery on a body area selected from the skin, scalp, hair and phanere, of a cosmetically effective amount of a cosmetic composition as defined in claim 15.

29. A method of cosmetic care comprising the topical delivery on a body area selected from the skin, scalp, hair and phanere, of a cosmetically effective amount of a cosmetic composition as defined in claim 16.

30. A method of cosmetic care comprising the topical delivery on a body area selected from the skin, scalp, hair and phanere, of a cosmetically effective amount of a cosmetic composition as defined in claim 17.
